(19) United States
(12) Patent Application Publication
(10) Pub. No.: US 2021/0180004 A1
(43) Pub. Date: Jun. 17, 2021

(54) CELL SORTING SYSTEM

(71) Applicant: WEINBERG MEDICAL PHYSICS INC., North Bethesda, MD (US)

(72) Inventors: Irving N. Weinberg, North Bethesda, MD (US); Roland PROBST, North Bethesda, MD (US)

(21) Appl. No.: 17/123,874

(22) Filed: Dec. 16, 2020

Related U.S. Application Data
(60) Provisional application No. 62/948,448, filed on Dec. 16, 2019.

(51) Int. Cl. C12M 1/00; C12M 1/30

(52) U.S. Cl. CPC C12M 47/04; C12M 33/02; C12M 31/10

(57) ABSTRACT

Devices and methods for sorting cells are provided that use a holder with cells adherent to a material whose adhesive properties are affected by light, a pixelated light-generating array, and a pixelated photosensitive array. Selected cells can be released from the adhesive layer after illumination of the adhesive layer by the light-generating array.

### PRIORITY

This patent application claims priority to U.S. Provisional Patent Application No. 62/948,448 entitled “CELL SORTING SYSTEM” filed Dec. 16, 2029, the disclosure of which being incorporated herein by reference in its entirety

### FIELD

Disclosed embodiments pertain to methodologies and equipment for a cell sorting.

### BACKGROUND

Stem cells or other cells may be characterized by shape. This characterization may be useful for determining which stem cells are likely to be successful when used in a clinical setting (for example: to form bone when placed in a human body part) or for determining which cells are best suited for further processing (for example: expansion of the cell line to produce more cells).

### SUMMARY

Disclosed embodiments provide devices and methods for processing cells and for manipulating cells a holder using a light-generating array.

### BRIEF DESCRIPTION OF THE FIGURES

FIG. 1 illustrates the apparatus (100), which includes an array (110) containing liquid crystals and light emitting diodes that can emit ultra-violet or other types of light from selected pixels in the array;

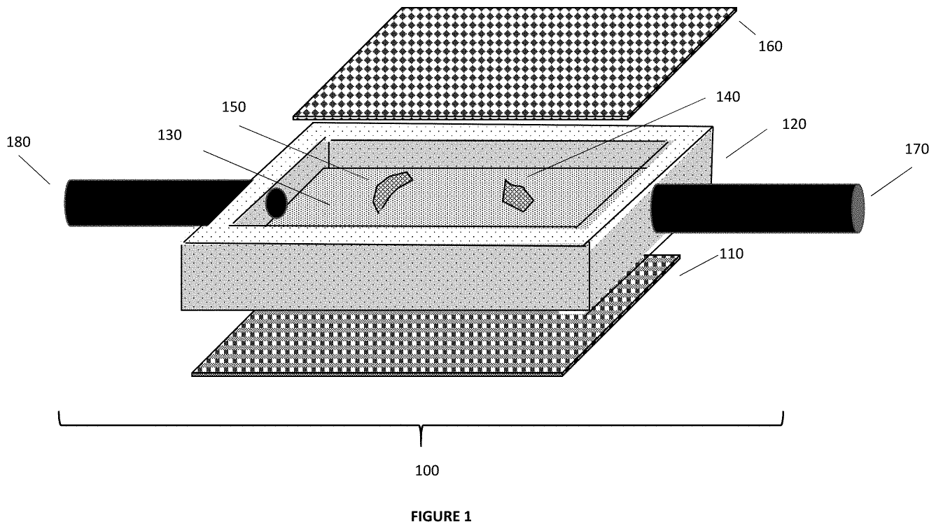

FIG. 2 shows the apparatus of FIG. 1, with one or more of the pixels in array (110) that have been activated to form a shape (200); and

FIG. 3 illustrates a method employed for cell processing.

### DETAILED DESCRIPTION

An apparatus and method for processing cells (140), (150) includes a holder (120) in which the cells can grow. It is understood that the term “holder” implies a chamber or any location in which cells can grow. The cells are affixed to holder (120) with a material (130) which is normally sticky but can be made less sticky through the application of light. The apparatus includes a light-sensitive array (160) for selecting cells based on the shape or images of the cells. The apparatus also includes an array (110) of light-generating pixels, for example an LCD/LED array similar to the ones used in LCD 3D printing. The apparatus includes photosensitive array (110) near or in holder (120). The photosensitive and light-generating arrays may include charge-coupled devices (CCD) that may be manufactured using complementary metal oxide semiconductor (CMOS) processes. The electronics that power and control the LCD/LED array (110) are not shown. Array (110) may be on one side of a holder (120) of cells. Holder (120) may be filled with fluid and/or cells and other materials. Light emitted from array (110) is transmissible through the base of holder (120). Holder (120) is coated on the inside (where the cells are) with a sticky substance (130) that binds cells to the holder. Cells (140) and (150) are shown by example resting on sticky substance (130). The sticky substance does not inhibit the growth or shape of cells (140) or (150) as they grow. Tubes or channels for instilling or removing fluids or cells from holder (120) are shown as (170) and (180). The pumping mechanisms or locations where the contents of (170) and (180) flow are not shown. A photosensitive array or other camera (160) is shown above the holder (120). It is understood that holder (120) may have a cover (not shown) that is able to transmit light from cells (iso) and (140) to the photosensitive array (160).

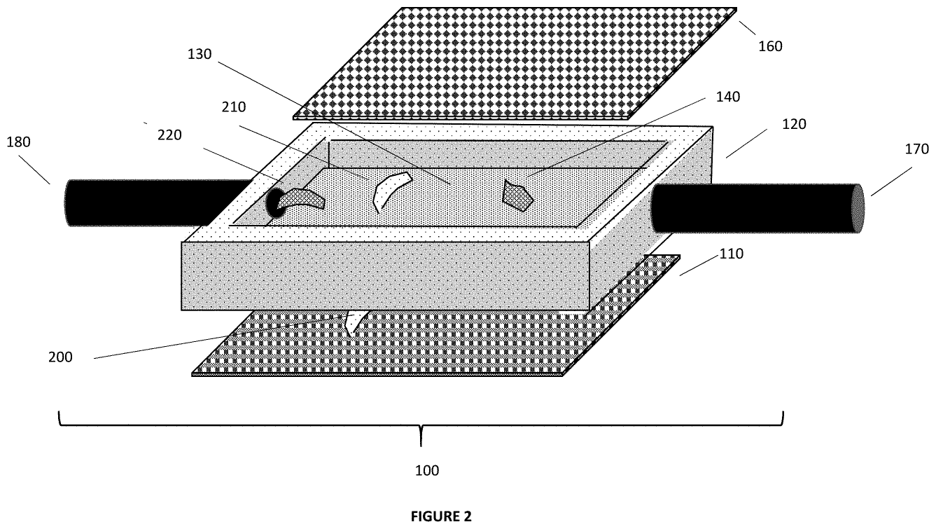

Activation of the array (110) emits ultraviolet or other light that changes the sticky properties of coating (130), represented in FIG. 2 by a region (210). For example, the light may have a wavelength and/or intensity to make cell (iso) no longer as adherent to coating (130) as before. Alternatively, the light may have a wavelength and/or intensity to make cell (iso) more adherent to coating (130) as compared to before the light. The cell (iso) that was in this region is no longer attached to the holder (120) and is free to be pulled into a channel (180) for further processing or disposal. Alternatively, a selected cell that was in this region may be preferentially attached to the holder (120) and other cells may be pulled into a channel (180) for further processing or disposal.

Light emitted from array (110), transmitted through holder (120) and impinging on photosensitive array (160) may be used to form an image of cell (150). This image formation may be performed using lensless techniques such as contact microscopy. The term “contact microscopy” is understood to included cases where the object of interest is near the photosensor, for example less than a centimeter, without being in actual contact. Alternatively, the light impinging on photosensitive array (160) may describe patterns correlated with the shape of cells without forming an image, for example by describing the Fourier spectrum generated by the light as it passed through cell (iso).

The description of cell (iso) determined by the pattern of light activating the photosensitive array (160) is used to determine whether to select a cell (iso) for removal from sticky layer (130). Alternatively, instead of using light from (110) to generate patterns on array (160), light may illuminate cells from other sources than array (110). Light from array (110) may be transmitted in a pattern through a portion of the base of holder (120) to act on the material (130) to change the sticky properties in a pattern corresponding to cells selected (for example, cell iso) so as to release selected cells as shown in FIG. 2. It is understood that the portion of the base of holder (120) may be a different material than the rest of the holder. For example, the portion may be a thin layer of mylar. Once released from material (130), cell (iso) may be removed from holder (120), for example by flowing fluid from channel (170) and into channel (180). Alternatively, holder (120) can be tipped to remove its fluid and any cells not adherent to holder (120).

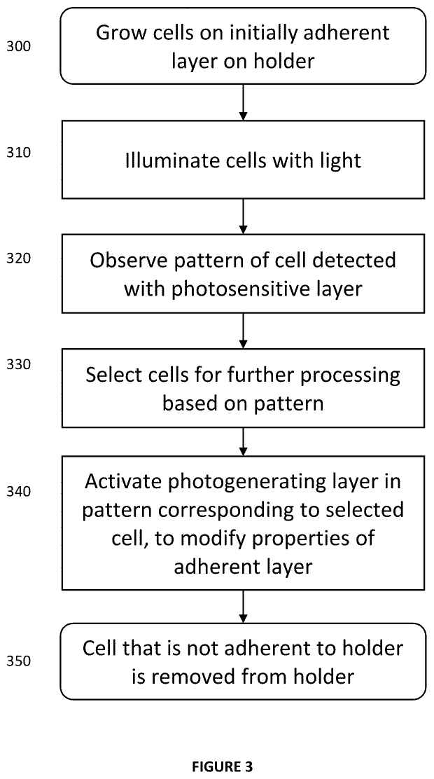

According to the method of FIG. 3, in a first step (300), cells (140) and (15o) are grown in a holder (120). In a subsequent step (310), cells are illuminated with light, for example from below with light-generating array (110) of from another source near or in holder (120). In subsequent step (320), the pattern of light detected with photosensitive layer (160) is analyzed with a computer (not shown). In subsequent step (330), the pattern is used to select whether specific cell (150) are to be subject to additional processing based. In subsequent step (340), pixels in, light-generating array (110) are selected in accordance with the pattern, and said pixels generate light of a wavelength and/or intensity that modifies the properties of layer (130) so that the adherence of cell (150) is different as compared to the case before the light was generated. In subsequent step (350), the cell or cells that are not adherent may be removed from the holder for further processing and/or disposal.

Light changes the properties of material (130), which may be a photo-responsive hydrogel, as described above. Examples of materials that get more sticky by solidifying are include sodium-alginate combined with a chelated form of calcium (Ca2+) and a photoacid generator (PAG). An example of a material that gets less sticky by liquifying is nitrobencyl ether-derived monomer reacted with poly(ethylene glycol)(PEG)-bis-amine. The gel degrades (liquifies) under cytocompatible irradiation conditions (365 nm to 420 nm) and has cytocompatible photochemistry. It has been used with live cell cultures, including human mesenchymal stem cells (hMSCs), with high viability measured by membrane integrity and DNA assays. Other materials whose properties change upon activation by light include: fibrin alginate, polyethylene glycol (PEG)-based hydrogel, alginate, alginate-lyase, gelatin norbornene (GelNB) and PEG, gelatin methacryloyl (GelMA), alginate/RGD-alginate, RGD-alginate, PEG-GelMA, Type I collagen- and chitosan-agarose blends, fibrin-collagen, agarose-collagen, methacrylamide gelatin, polylactic acid (PLA)/GelMA, cellulose and alginate, skin-derived ECM, polyester urethane urea (PEUU), plasma-alginate, albumin, chitosan-alginate, polystyrene, sodium-alginate, chelated forms of calcium (Ca2+), nitrobenzyl ether derived monomer, calcium-chelator such as ethylene diamine tetracetic acid (EDTA) to convert the gel into a solution, PEG-bis-amine to form a photodegradable cross-linker, fibronectin-derived peptide RGDS to form a photoreleasable tether, and photolabile group ethyl 4-(4-(1-hydroxyethyl)-2-methoxy-5-nitrophenoxy)butanoic acid.

The term “sticky” is understood to mean relatively adherent. “Less sticky” is understood to mean less adherent. The physical property of material (130) may also be described in terms of “wettability”, which affects the adherent property.

For the purposes of this disclosure, “near” implies less than 10 centimeters from the cells. It is understood that the term “cell” (150) may represent an assembly or multiplicity of cells or an organism (for example a worm). It is understood that cell (150) may be an unwanted cell or may be a desired type of cell. It is understood that holder (120) may move between light-generating pixelated array and photosensitive array (160) to assist in describing the shapes of cells, for example by tomographic effect as described in U.S. Pat. No. 8,306,179, entitled “Reconstruction of linearly moving objects with intermittent x-ray sources”, but in the case of this invention the x-ray sources would be replaced by light sources. It is understood that the term “light” implies electromagnetic radiation of any wavelength or combination of wavelengths.

1. An apparatus comprising a holder of cells, wherein cells are adherent to the holder with a material whose adhesive properties are affected by light, a pixelated light-generating array, and a pixelated photosensitive array, and at least one port for removing the contents of the holder.

2. The apparatus of claim 1, where the adhesive material is a photo-responsive hydrogel.

3. The apparatus of claim 2, wherein the photo-responsive hydrogel is composed of one or more of the following materials
a. Fibrin Alginate
b. Polyethylene glycol (PEG)-based hydrogel
c. Alginate
d. Alginate-lyase
e. Gelatin norbornene (GelNB) and PEG
f. Gelatin methacryloyl (GelMA)
g. Alginate/RGD-alginate
h. RGD-alginate
i. PEG-GelMA
j. Type I collagen- and chitosan-agarose blends
k. Fibrin-collagen
l. Agarose-collagen
m. Methacrylamide gelatin
n. Polylactic acid (PLA)/GelMA
o. Cellulose and alginate
p. Skin-derived ECM
q. Polyester urethane urea (PEUU)
r. Plasma-alginate
s. Albumin
t. Chitosan-alginate
u. Polystyrene
v. Sodium-alginate
w. Chelated form of calcium (Ca2+)
x. Nitrobenzyl ether derived monomer
y. Calcium-chelator such as ethylene diamine tetracetic acid (EDTA) to convert the gel into a solution
z. PEG-bis-amine to form a photodegradable cross-linker
aa. Fibronectin-derived peptide RGDS to form a photoreleasable tether
bb. Photolabile group, ethyl 4-(4-(1-hydroxyethyl)-2-methoxy-5-nitrophenoxy)butanoic acid

4. The apparatus of claim 1, where the adhesive material is a polymer film that changes its wettability upon ultraviolet irradiation.

5. The apparatus of claim 1, where light increases the adhesive properties of the material.

6. The apparatus of claim 1, where light decreases the adhesive properties of the material.

7. The apparatus of claim 1, where the light-generating array includes liquid crystal and/or light emitting diodes.

8. The apparatus of claim 11, where the photosensitive array includes conventional CCD or CMOS sensors.

9. The apparatus of claim 1, where the photosensitive array includes lensless contact imagers.

10. The apparatus of claim 1, where the photosensitive array includes very-large-area CCD image sensors.

11. A method for processing cells, in which selected cells are released from an initially adhesive layer in a chamber after illumination of said adhesive layer from a light-generating array and then transported after release to another chamber.

12. The method of claim 10, where the pattern of illumination is selected on the basis of a pattern or patterns formed on a photosensitive layer in or near the chamber.
